SEQUENCE LISTING (3 sequences)

SEQ ID NO: 1 - protein, 9 aa, synthetic construct
CGLMVGGVV

SEQ ID NO: 2 - protein, 3414 aa, Megathura crenulata
MLSVRLLIVVLALANAENLVRKSVEHLTQEETLDLQAALRELQMDSSSIGFQKIAAAHGAPASCVHKDTSIACCIHGMPTFPHWHRAYVVHMERALQTKRRTSGLPYWDWTEPITQLPSLAADPVYIDSQGGKAHTNYWYRGNIDFLDKKTNRAVDDRLFEKVKPGQHTHLMESVLDALEQDEFCKFEIQFELAHNAIHYLVGGKHDYSMANLEYTAYDPIFFLHHSNVDRIFAIWQRLQELRNKDPKAMDCAQELLHQKMEPFSWEDNDIPLTNEHSTPADLFDYCELHYDYDTLNLNGMTPEELKTYLDERSSRARAFASFRLKGFGGSANVFVYVCIPDDNDRNDDHCEKAGDFFVLGGPSEMKWQFYRPYLFDLSDTVHKMGMKLDGHYTVKAELFSVNGTALPDDLLPHPVVVHHPEKGFTDPPVKHHQSANLLVRKNINDLTREEVLNLREAFHKFQEDRSVDGYQATAEYHGLPARCPRPDAKDRYACCVHGMPIFPHWHRLFVTQVEDALVGRGATIGIPYWDWTEPMTHIPGLAGNKTYVDSHGASHTNPFHSSVIAFEENAPHTKRQIDQRLFKPATFGHHTDLFNQILYAFEQEDYCDFEVQFEITHNTIHAWTGGSEHFSMSSLHYTAFDPLFYFHHSNVDRLWAVWQALQMRRHKPYRAHCAISLEHMHLKPFAFSSPLNNNEKTHANAMPNKIYDYENVLHYTYEDLTFGGISLENIEKMIHENQQEDRIYAGFLLAGIRTSANVDIFIKTTDSVQHKAGTFAVLGGSKEMKWGFDRVFKFDITHVLKDLDLTADGDFEVTVDITEVDGTKLASSLIPHASVIREHARVKFDKVPRSRLIRKNVDRLSPEEMNELRKALALLKEDKSAGGFQQLGAFHGEPKWCPSPEASKKFACCVHGMSVFPHWHRLLTVQSENALRRHGYDGALPYWDWTSPLNHLPELADHEKYVDPEDGVEKHNPWFDGHIDTVDKTTTRSVQNKLFEQPEFGHYTSIAKQVLLALEQDNFCDFEIQYEIAHNYIHALVGGAQPYGMASLRYTAFDPLFYLHHSNTDRIWAIWQALQKYRGKPYNVANCAVTSMREPLQPFGLSANINTDHVTKEHSVPFNVFDYKTNFNYEYDTLEFNGLSISQLNKKLEAIKSQDRFFAGFLLSGFKKSSLVKFNICTDSSNCHPAGEFYLLGDENEMPWAYDRVFKYDITEKLHDLKLHAEDHFYIDYEVFDLKPASLGKDLFKQPSVIHEPRIGHHEGEVYQAEVTSANRIRKNIENLSLGELESLRAAFLEIENDGTYESIAKFHGSPGLCQLNGNPISCCVHGMPTFPHWHRLYVVVVENALLKKGSSVAVPYWDWTKRIEHLPHLISDATYYNSRQHHYETNPFHHGKITHENEITTRDPKDSLFHSDYFYEQVLYALEQDNFCDFEIQLEILHNALHSLLGGKGKYSMSNLDYAAFDPVFFLHHATTDRIWAIWQDLQRFRKRPYREANCAIQLMHTPLQPFDKSDNNDEATKTHATPHDGFEYQNSFGYAYDNLELNHYSIPQLDHMLQERKRHDRVFAGFLLHNIGTSADGHVFVCLPTGEHTKDCSHEAGMFSILGGQTEMSFVFDRLYKLDITKALKKNGVHLQGDFDLEIEITAVNGSHLDSHVIHSPTILFEAGTDSAHTDDGHTEPVMIRKDITQLDKRQQLSLVKALESMKADHSSDGFQAIASFHALPPLCPSPAASKRFACCVHGMATFPQWHRLYTVQFQDSLRKHGAVVGLPYWDWTLPRSELPELLTVSTIHDPETGRDIPNPFIGSKIEFEGENVHTKRDINRDRLFQGSTKTHHNWFIEQALLALEQTNYCDFEVQFEIMHNGVHTWVGGKEPYGIGHLHYASYDPLFYIHHSQTDRIWAIWQSLQRFRGLSGSEANCAVNLMKTPLKPFSFGAPYNLNDHTHDFSKPEDTFDYQKFGYIYDTLEFAGWSIRGIDHIVRNRQEHSRVFAGFLLEGFGTSATVDFQVCRTAGDCEDAGYFTVLGGEKEMPWAFDRLYKYDITETLDKMNLRHDEIFQIEVTITSYDGTVLDSGLIPTPSIIYDPAHHDISSHHLSLNKVRHDLSTLSERDIGSLKYALSSLQADTSADGFAAIASFHGLPAKCNDSHNNEVACCIHGMPTFPHWHRLYTLQFEQALRRHGSSVAVPYWDWTKPIHNIPHLFTDKEYYDVWRNKVMPNPFARGYVPSHDTYTVRDVQEGLFHLTSTGEHSALLNQALLALEQHDYCDFAVQFEVMHNTIHYLVGGPQVYSLSSLHYASYDPIFFIHHSFVDKVWAVWQALQEKRGLPSDRADCAVSLMTQNMRPFHYEINHNQFTKKHAVPNDVFKYELLGYRYDNLEIGGMNLHEIEKEIKDKQHHVRVFAGFLLHGIRTSADVQFQICKTSEDCHHGGQIFVLGGTKEMAWAYNRLFKYDITHALHDAHITPEDVFHPSEPFFIKVSVTAVNGTVLPASILHAPTIIYEPGLDHHEDHHSSSMAGHGVRKEINTLTTAEVDNLKDAMRAVMADHGPNGYQAIAAFHGNPPMCPMPDGKNYSCCTHGMATFPHWHRLYTKQMEDALTAHGARVGLPYWDGTTAFTALPTFVTDEEDNPFHHGHIDYLGVDTTRSPRDKLFNDPERGSESFFYRQVLLALEQTDFCQFEVQFEITHNAIHSWTGGLTPYGMSTLEYTTYDPLFWLHHANTDRIWAIWQALQEYRGLPYDHANCEIQAMKRPLRPFSDPINHNAFTHSNAKPTDVFEYSRFNFQYDNLRFHGMTIKKLEHELEKQKEEDRTFAAFLLHGIKKSADVSFDVCNHDGECHFAGTFAILGGEHEMPWSFDRLFRYDITQVLKQMHLEYDSDFTFHMRIIDTSGKQLPSDLIKMPTVEHSPGGKHHEKHHEDHHEDILVRKNIHSLSHHEAEELRDALYKLQNDESHGGYEHIAGFHGYPNLCPEKGDEKYPCCVHGMSIFPHWHRLHTIQFERALKKHGSHLGIPYWDWTQTISSLPTFFADSGNNNPFFKYHIRSINQDTVRDVNEAIFQQTKFGEFSSIFYLALQALEEDNYCDFEVQYEILHNEVHALIGGAEKYSMSTLEYSAFDPYFMIHHASLDKIWIIWQELQKRRVKPAHAGSCAGDIMHVPLHPFNYESVNNDDFTRENSLPNAVVDSHRFNYKYDNLNLHGHNIEELEEVLRSLRLKSRVFAGFVLSGIRTTAVVKVYIKSGTDSDDEYAGSFVILGGAKEMPWAYERLYRFDITETVHNLNLTDDHVKFRFDLKKYDHTELDASVLPAPIIVRRPNNAVFDIIEIPIGKDVNLPPKVVVKRGTKIMFMSVDEAVTTPMLNLGSYTAMFKCKVPPFSFHAFELGKMYSVESGDYFMTASTTELCNDNNLRIHVHVDDE

SEQ ID NO: 3 - protein, 3423 aa, synthetic construct
MLSVRLLIVVLALANAENLVRKSVEHLTQEETLDLQAALRELQMDSSSIGFQKIAAAHGAPASCVHKDTSIACCIHGMPTFPHWHRAYVVHMERALQTKRRTSGLPYWDWTEPITQLPSLAADPVYIDSQGGKAHTNYWYRGNIDFLDKKTNRAVDDRLFEKVKPGQHTHLMESVLDALEQDEFCKFEIQFELAHNAIHYLVGGKHDYSMANLEYTAYDPIFFLHHSNVDRIFAIWQRLQELRNKDPKAMDCAQELLHQKMEPFSWEDNDIPLTNEHSTPADLFDYCELHYDYDTLNLNGMTPEELKTYLDERSSRARAFASFRLKGFGGSANVFVYVCIPDDNDRNDDHCEKAGDFFVLGGPSEMKWQFYRPYLFDLSDTVHKMGMKLDGHYTVKAELFSVNGTALPDDLLPHPVVVHHPEKGFTDPPVKHHQSANLLVRKNINDLTREEVLNLREAFHKFQEDRSVDGYQATAEYHGLPARCPRPDAKDRYACCVHGMPIFPHWHRLFVTQVEDALVGRGATIGIPYWDWTEPMTHIPGLAGNKTYVDSHGASHTNPFHSSVIAFEENAPHTKRQIDQRLFKPATFGHHTDLFNQILYAFEQEDYCDFEVQFEITHNTIHAWTGGSEHFSMSSLHYTAFDPLFYFHHSNVDRLWAVWQALQMRRHKPYRAHCAISLEHMHLKPFAFSSPLNNNEKTHANAMPNKIYDYENVLHYTYEDLTFGGISLENIEKMIHENQQEDRIYAGFLLAGIRTSANVDIFIKTTDSVQHKAGTFAVLGGSKEMKWGFDRVFKFDITHVLKDLDLTADGDFEVTVDITEVDGTKLASSLIPHASVIREHARVKFDKVPRSRLIRKNVDRLSPEEMNELRKALALLKEDKSAGGFQQLGAFHGEPKWCPSPEASKKFACCVHGMSVFPHWHRLLTVQSENALRRHGYDGALPYWDWTSPLNHLPELADHEKYVDPEDGVEKHNPWFDGHIDTVDKTTTRSVQNKLFEQPEFGHYTSIAKQVLLALEQDNFCDFEIQYEIAHNYIHALVGGAQPYGMASLRYTAFDPLFYLHHSNTDRIWAIWQALQKYRGKPYNVANCAVTSMREPLQPFGLSANINTDHVTKEHSVPFNVFDYKTNFNYEYDTLEFNGLSISQLNKKLEAIKSQDRFFAGFLLSGFKKSSLVKFNICTDSSNCHPAGEFYLLGDENEMPWAYDRVFKYDITEKLHDLKLHAEDHFYIDYEVFDLKPASLGKDLFKQPSVIHEPRIGHHEGEVYQAEVTSANRIRKNIENLSLGELESLRAAFLEIENDGTYESIAKFHGSPGLCQLNGNPISCCVHGMPTFPHWHRLYVVVVENALLKKGSSVAVPYWDWTKRIEHLPHLISDATYYNSRQHHYETNPFHHGKITHENEITTRDPKDSLFHSDYFYEQVLYALEQDNFCDFEIQLEILHNALHSLLGGKGKYSMSNLDYAAFDPVFFLHHATTDRIWAIWQDLQRFRKRPYREANCAIQLMHTPLQPFDKSDNNDEATKTHATPHDGFEYQNSFGYAYDNLELNHYSIPQLDHMLQERKRHDRVFAGFLLHNIGTSADGHVFVCLPTGEHTKDCSHEAGMFSILGGQTEMSFVFDRLYKLDITKALKKNGVHLQGDFDLEIEITAVNGSHLDSHVIHSPTILFEAGTDSAHTDDGHTEPVMIRKDITQLDKRQQLSLVKALESMKADHSSDGFQAIASFHALPPLCPSPAASKRFACCVHGMATFPQWHRLYTVQFQDSLRKHGAVVGLPYWDWTLPRSELPELLTVSTIHDPETGRDIPNPFIGSKIEFEGENVHTKRDINRDRLFQGSTKTHHNWFIEQALLALEQTNYCDFEVQFEIMHNGVHTWVGGKEPYGIGHLHYASYDPLFYIHHSQTDRIWAIWQSLQRFRGLSGSEANCAVNLMKTPLKPFSFGAPYNLNDHTHDFSKPEDTFDYQKFGYIYDTLEFAGWSIRGIDHIVRNRQEHSRVFAGFLLEGFGTSATVDFQVCRTAGDCEDAGYFTVLGGEKEMPWAFDRLYKYDITETLDKMNLRHDEIFQIEVTITSYDGTVLDSGLIPTPSIIYDPAHHDISSHHLSLNKVRHDLSTLSERDIGSLKYALSSLQADTSADGFAAIASFHGLPAKCNDSHNNEVACCIHGMPTFPHWHRLYTLQFEQALRRHGSSVAVPYWDWTKPIHNIPHLFTDKEYYDVWRNKVMPNPFARGYVPSHDTYTVRDVQEGLFHLTSTGEHSALLNQALLALEQHDYCDFAVQFEVMHNTIHYLVGGPQVYSLSSLHYASYDPIFFIHHSFVDKVWAVWQALQEKRGLPSDRADCAVSLMTQNMRPFHYEINHNQFTKKHAVPNDVFKYELLGYRYDNLEIGGMNLHEIEKEIKDKQHHVRVFAGFLLHGIRTSADVQFQICKTSEDCHHGGQIFVLGGTKEMAWAYNRLFKYDITHALHDAHITPEDVFHPSEPFFIKVSVTAVNGTVLPASILHAPTIIYEPGLDHHEDHHSSSMAGHGVRKEINTLTTAEVDNLKDAMRAVMADHGPNGYQAIAAFHGNPPMCPMPDGKNYSCCTHGMATFPHWHRLYTKQMEDALTAHGARVGLPYWDGTTAFTALPTFVTDEEDNPFHHGHIDYLGVDTTRSPRDKLFNDPERGSESFFYRQVLLALEQTDFCQFEVQFEITHNAIHSWTGGLTPYGMSTLEYTTYDPLFWLHHANTDRIWAIWQALQEYRGLPYDHANCEIQAMKRPLRPFSDPINHNAFTHSNAKPTDVFEYSRFNFQYDNLRFHGMTIKKLEHELEKQKEEDRTFAAFLLHGIKKSADVSFDVCNHDGECHFAGTFAILGGEHEMPWSFDRLFRYDITQVLKQMHLEYDSDFTFHMRIIDTSGKQLPSDLIKMPTVEHSPGGKHHEKHHEDHHEDILVRKNIHSLSHHEAEELRDALYKLQNDESHGGYEHIAGFHGYPNLCPEKGDEKYPCCVHGMSIFPHWHRLHTIQFERALKKHGSHLGIPYWDWTQTISSLPTFFADSGNNNPFFKYHIRSINQDTVRDVNEAIFQQTKFGEFSSIFYLALQALEEDNYCDFEVQYEILHNEVHALIGGAEKYSMSTLEYSAFDPYFMIHHASLDKIWIIWQELQKRRVKPAHAGSCAGDIMHVPLHPFNYESVNNDDFTRENSLPNAVVDSHRFNYKYDNLNLHGHNIEELEEVLRSLRLKSRVFAGFVLSGIRTTAVVKVYIKSGTDSDDEYAGSFVILGGAKEMPWAYERLYRFDITETVHNLNLTDDHVKFRFDLKKYDHTELDASVLPAPIIVRRPNNAVFDIIEIPIGKDVNLPPKVVVKRGTKIMFMSVDEAVTTPMLNLGSYTAMFKCKVPPFSFHAFELGKMYSVESGDYFMTASTTELCNDNNLRIHVHVDDECGLMVGGVV

(19) United States
(12) Patent Application Publication
(10) Pub. No.: US 2026/0273032 A1
(43) Pub. Date: Sep. 17, 2026

(54) VACCINATION AGAINST THE AMYLOID-BETA 40 PEPTIDE

(71) Applicant: ARACLON BIOTECH, S.L., Zaragoza (ES)

(72) Inventors: Jose Manuel Sarasa Barrio, Zaragoza (ES); Jose Vicente Terencio Alemany, Sant Cugat Del Valles (ES); Jorge Loscos Aranda, Zaragoza (ES); Maria Rafaela Pascual Lucas, Zaragoza (ES); Jesus Luis Canudas Becana, Zaragoza (ES); Maria Montanes Bellosta, Zaragoza (ES); Ana Maria Lacosta Lopez-Alda, Zaragoza (ES)

(21) Appl. No.: 19/580,953

(22) PCT Filed: Oct. 21, 2024
(86) PCT No.: PCT/EP2024/079709

§ 371 Date: Apr. 22, 2026

(30) Foreign Application Priority Data
Oct. 23, 2023 (EP) 23383081.9

(51) Int. Cl. A61K 39/00; A61K 39/385; A61K 45/06; A61P 25/28

(52) U.S. Cl. CPC A61K 39/0007; A61K 39/385; A61K 45/06; A61P 25/28; A61K 2039/54; A61K 2039/545; A61K 2039/55505; A61K 2039/575; A61K 2039/6081

(57) ABSTRACT

The present invention refers to a vaccine composition comprising amyloid beta 33-40 (Aβ33-40) peptide with an additional cysteine in the N-terminal end (CysAβ33-40) conjugated with the carrier protein keyhole limpet hemocyanin (KLH) using as the crosslinking agent the maleimidobutiric acid N-hydroxysuccinimide ester (SM) and aluminium hydroxide gel as adjuvant for the treatment and/or prevention of cerebral amyloid angiopathy (CAA), the method comprising administering a therapeutically effective amount of said vaccine composition comprising to a patient in need thereof.

### FIELD OF THE INVENTION

The present invention relates to the field of medicine and, in particular to the treatment of neural pathologies, such as Alzheimer's disease (AD), cerebral amyloid angiopathy (CAA), or mild cognitive impairment (MCI), using a vaccine against the amyloid-beta 40 peptide. In particular, the vaccine composition is comprised by the amyloid beta 33-40 (Aβ33-40) peptide with an additional cysteine in the N-terminal end (CysAβ33-40) conjugated with the carrier protein keyhole limpet hemocyanin (KLH) using as the crosslinking agent the maleimidobutiric acid N-hydroxysuccinimide ester (SM) and aluminium hydroxide gel as adjuvant.

### Background of the Invention

AD is the most common type of dementia, accounting for 50-75% of the estimated 47 million people with dementia worldwide. The amyloid cascade hypothesis of AD proposes that amyloid-β (Aβ) peptide accumulation in the brain, caused by an imbalance between Aβ production and clearance, is the initiating factor of a cascade ultimately leading to dementia.

Aβ peptides are generated from sequential cleavage of the amyloid precursor protein (APP), including Aβ40 and Aβ42. Aβ40 is the predominant variant (90%) among the secreted Aβ forms and although Aβ42 is more hydrophobic and prone to aggregate, and Aβ42 oligomers are regarded to be the most neurotoxic species, Aβ40 can also produce highly toxic diffusible aggregates, which can be prevented in vitro by specific anti-Aβ40 antibodies.

Several studies have proposed that different concentrations of Aβ40 in the brain distinguishes patients with AD from those who have senile plaques but are cognitively normal, pointing to the importance of Aβ40 in the onset of dementia. In addition, Aβ40 is the main component of amyloid deposition around cerebral arteries causing cerebral amyloid angiopathy (CAA), which has a prevalence of about 80-90% in patients with AD. CAA is a nonspecific disease entity that has been associated with a number of neuropathological conditions which is characterized by the pathologic accumulation of Aβ plaques in the tunica media and tunica adventitia of small and mid-sized arteries, and less frequently of veins, of the cerebral cortex and the leptomeninges resulting in vascular fragility, intracranial bleeding (lobar intracerebral hemorrhage) and in some cases dementia. Currently, there is no known effective treatment to decrease or prevent the underlying deposition of Aβ that characterizes CAA.

Considering those previous results suggesting that strategies targeting Aβ40 could represent novel disease-modifying therapies, the present inventors have developed ABvac40, the first active vaccine targeting the C-terminal end of the Aβ40 peptide. ABvac40 consists in a conjugate of Aβ33-40 peptide with an additional cysteine in the N-terminal end (CysAβ33-40) conjugated to the carrier protein keyhole limpet hemocyanin (KLH) using as the crosslinking agent the maleimidobutiric acid N-hydroxysuccinimide ester (SM) and aluminium hydroxide gel as adjuvant.

In a Phase II study, safety and tolerability of the vaccine in patients with Amnestic Mild Cognitive Impairment (a-MCI) or Very Mild Alzheimer's Disease (vm-AD) was confirmed, and the present inventors has surprisingly found that plasma antibodies triggered by said vaccine recognize the C-terminal region of Aβx-40 peptides in different aggregated states (monomeric, oligomeric, and brain deposits), and the fact that binding of vaccine-elicited antibodies to vascular and parenchymal amyloid brain deposits could lead to a reduction of amyloid deposition in cerebral vessels. This could enhance the clearance of brain waste through perivascular spaces, potentially contributing to cognitive improvement, while maintaining an excellent safety profile of the vaccine.

### DESCRIPTION OF THE INVENTION

The words “comprises/comprising” and the words “having/including” when used herein with reference to the present invention are used to specify the presence of stated features, integers, steps or components but do not preclude the presence or addition of one or more other features, integers, steps, components, or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

As used herein, “amyloid disease” and the plural thereof refer to diseases associated with the β-amyloid accumulation. Said accumulation can fundamentally occur in the brain, producing diseases among which are found AD, Parkinson's disease, CAA, vascular dementia of an amyloid origin and dementia with Lewy bodies. β-amyloid accumulation can also fundamentally occur in skeletal muscle, producing inclusion-body myositis.

As used herein, “active immunization” and the plural thereof refer to the delivery to a patient of immunogens, i.e., they allow antibody generation in said patient.

As used herein, “adjuvant” and the plural thereof refer to immunomodulatory substances capable of being combined with the conjugate of the present invention for increasing, improving or otherwise modulating an immune response in a patient.

As used herein, “patient” and the plural thereof refer to any mammal, preferably human, in which the conjugate of the present invention or a composition comprising same can be administered for the purpose of treating or preventing an amyloid disease.

As used herein, “Aβ(33-40)” and “CysAβ(33-40)” are used indistinctively and refer to a synthetic peptide corresponding to the sequence of positions 33 to 40 of amyloid beta 40 with an additional cysteine (C) in the N-terminal end. Said sequence is set forth in SEQ ID NO: 1 and is: CGLMVGGVV.

As used herein, “crosslinking agent” refers to any chemical or biochemical component that allows for two elements to be chemically linked. In this particular case, it is used for the linkage between the carrier protein KLH and the main immunogenic peptide CysAβ33-40.

As used herein, KLH refers to the keyhole limpet hemocyanin protein, a highly immunogenic metalloprotein commonly used as a carrier in many vaccines and conjugates, and its amino acid sequence is set forth in SEQ ID NO: 2.

As used herein, “cerebral amyloid angiopathy (CAA)” refers to a type of cerebrovascular disorder characterized by the accumulation of amyloid beta-peptide within the leptomeninges and small/medium-sized cerebral blood vessels, which results in fragile vessels that may manifest in lobar intracerebral hemorrhages.

As used herein, the term “vaccine” refers to any preparation that is used to stimulate the body's immune response against diseases.

Finally, as used herein the term “ABvac40 vaccine” refers to the composition to the present invention, and comprises a conjugate of Aβ33-40 with an additional cysteine in the N-terminal end (CysAβ33-40) and a carrier protein (KLH) using as the crosslinking agent the maleimidobutiric acid N-hydroxysuccinimide ester (SM) and containing aluminium hydroxide as adjuvant. The amino acid sequence of the ABvac 40 vaccine is shown in FIG. 1 and is set forth in SEQ ID NO: 3. It should be noted that the final sequence of the conjugate may vary, as multiple peptides will be conjugated via different lysine amino acids of the KLH protein. The ABvac40 vaccine of the present invention is prepared by firstly binding the crosslinking agent, in this case SM, to KLH, which is called KLH activation step. In a second step, the amount of the CysAβ(33-40) immunogenic peptide in dimethyl sulfoxide DMSO is calculated and added to the activated KLH-SM mix, so that conjugation can take place. After the conjugation has ended, the excess non-conjugated KLH and CysAβ(33-40) may be removed, while the conjugated peptide is to be stored at 4° C. until its use.

Methods of Prevention and/or Treatment

In a first aspect, the present invention provides a vaccine composition for use in the prevention and/or treatment of cerebral amyloid angiopathy (CAA), said vaccine composition comprising an amyloid beta 33-40 (Aβ33-40) peptide with an additional cysteine on the N-terminal end (Cys Aβ33-40) conjugated with the carrier keyhole limpet hemocyanin (KLH), using as the crosslinking agent the maleimidobutiric acid N-hydroxysuccinimide ester (SM) and an aluminum hydroxide gel.

The preparation of such a vaccine composition as used in the method of the invention can be performed using routine methods in the art. Moreover the sequence of the amyloid beta 33-40 (Aβ33-40) peptide with an additional cysteine on the N-terminal end (Cys Aβ33-40) is provided herein. As way of demonstration, Example 1 below provides a protocol for the preparation of a vaccine composition which can be used in the present invention.

In one embodiment, the aluminum hydroxide gel is Al(OH)₃. In one preferred embodiment Al(OH)₃ is at a concentration is between 0.1% and 1% (w/v), more preferably between 0.25 and 0.50% (w/v).

In one embodiment, the vaccine composition comprises the CysAβ33-40 peptide at a concentration between 50 μg/ml and 500 μg/ml. More preferably, it is at a concentration between 75 μg/ml and 400 μg/ml, and more preferably at a concentration between 100 μg/ml and 300 μg/ml. In a more preferred embodiment, the concentration of the CysAβ33-40 peptide in the vaccine is 200 μg/ml.

In one embodiment, the vaccine composition is administered by an administration route selected from the group consisting of intravenous, subcutaneous, intramuscular, intradermal, intraperitoneal, intrapulmonary, intranasal, oral, rectal, and combinations thereof. In a preferred embodiment, the vaccine composition is administered intravenously or subcutaneously. In a most preferred embodiment, the vaccine composition is administered subcutaneously. These routes of administration are well known in the art and can be readily adopted by the skilled person.

The vaccine composition for use in the present invention may be administered according to typical vaccination regimes, ranging from single doses to multiple doses separated by varying amounts of time.

However in a preferred embodiment, the vaccine composition of is administered at multiple intervals as part of a multiple-dosage regimen. In a preferred embodiment, the multiple dosing regimen comprises multiple portion doses administered on weeks 0, 4, 8, 12 and 16. In a more preferred embodiment, a booster is administered on week 42. In an even more preferred embodiment, a second booster is administered on week 58. As can be seen in the accompanying Examples, such an administration regime provided surprising and unanticipated efficacy in the prevention and/or treatment of CAA.

The vaccine composition for use in the present invention may be administered in varying quantities, according to typical vaccination amounts.

However, in a preferred embodiment the vaccine composition is administered at individual doses of about 0.5 ml to about 2 ml (0.2 mg/ml of CysAβ33-40) per interval as part of a multiple-dosage regimen. In a more preferred embodiment the vaccine composition is administered at individual doses of about 1 ml. As can be seen in the accompanying Examples, this amount of vaccine as used in the preferred administration regime provided surprising and unanticipated efficacy in the prevention and/or treatment of CAA.

In one embodiment, the vaccine composition is administered with supplementary active compounds. In a preferred embodiment, the supplementary active compounds are plasma-based proteins. In an even more preferred embodiment, said supplementary active compounds are albumin, alpha-1 antitrypsin/alpha-1 proteinase inhibitor, antithrombin, polyclonal immunoglobulins (such as IgG, IgA or combinations thereof), polyspecific immunoglobulins (such as IgM), C1 esterase inhibitor, thansthyretin, and combinations thereof.

In an alternative embodiment, the supplementary active compounds are neurogenic and/or neurotrophic compounds. In a preferred embodiment, said supplementary active compounds are brain-derived neurotrophic factor, glial cell line-derived neurotrophic factor, cilliary neurotrophic factor 1, amino acids 1-38 of pituitary adenylate cyclase-activating polypeptide, Y-27632 and pharmaceutically acceptable salts thereof [trans-4-[(1R)-1-Aminoethyl]-N-4-pyridinylcyclohexanecarboxamide], fasudil and pharmaceutically acceptable salts thereof [hexahydro-1-(5-isoquinolinyl-sulfonyl)-1H-1,4-diazepine], and combinations thereof. In an even more preferred embodiment, said supplementary active compounds are a combination of plasma-based products and neurogenic and/or neurotrophic compounds.

Supplementary active compounds also include medicaments for the prevention and/or treatment of Alzheimer's Disease (AD). For example, donepezil (aricept), galantamine (razadyne), rivastigmine (exelon), memantine (namenda), as well as other medicaments developed for this purpose.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g. in light of a history of symptoms and/or in light of genetic or other susceptibility factors). Treatment may also be continued after symptoms have resolved, for example to prevent or delay their recurrence. In some embodiments the methods provided herein refer to the treatment of a subject having CAA (e.g., a human CAA patient).

The vaccine composition for use in the invention may be administered to any subject for the prevention and/or treatment of CAA.

However it is preferred that the composition for use in the invention is administered to subjects who have developed, or are at risk of developing, CAA. Such subjects can be characterized as subjects from 50 years old and onwards, preferably between 55 and 80 years, regardless of the gender, that show any early symptoms of CAA, or that have an unusual score in any of the more common cognitive tests:
Mini-Mental State Examination (MMSE) between 24 and 30.
Clinical Dementia Rating (CDR) Scale of 0.5.
Repeatable Battery for the Assessment of Neuropsychological Status (RBANS) of 85 or lower.
Trail Making Test (TMT, consists of two parts, TMT-A and TMT-B).
Patients that have a family history, as there are cases of hereditary CAA, predominantly autosomal dominant mutations in the APP gene, that usually show signs earlier than those who suffer from sporadic CAA.
Patients that have already been diagnosed with CAA or any type of amyloid-related pathology, where ABvac40 can be used as a treatment.

A further aspect of the present invention is wherein the vaccine composition of the invention is used in combination of one or more further vaccines that have been developed for the prevention and/or treatment of CAA or other associated neurological disorders, in particular AD.

AD vaccination has been the subject of much clinical development in recent years. At the present time several vaccines are under review and may receive clinical authorization in the near future, including UB-311 which is vaccine under development by Vaxxinity, ACI-24.060 which is a vaccine under development by AC Immune, and ALZ-101 which is a vaccine under development by Alzinova. Further vaccines under development include ACI-35.030, which is a vaccine under development by AC Immune, AADvac1 which is a vaccine under development by Axon Neuroscience, and a tuberculosis vaccine called Bacillus Calmette-Guérin (BCG), and Tdap (tetanus toxoid, reduced diphtheria toxoid and acellular pertussis) vaccination.

Additional vaccines which may be combined with vaccine composition of the invention include AN1792, AIP-001 (Elan/Wyeth), CAD106 (Novartis), ACC001 (Pfizer), Affitope AD02 (Affiriis AG), Aducanumab/Aduhelm (Biogen/Neurimmune), Lecanemab/Leqembi (Eisai/Biogen), Donanemab/Kisunla (Eli Lilly), Solanezumab (Eli Lilly).

As used herein, the term “combination,” in the context of the administration of one or more vaccines does not restrict the order in which the vaccines to a subject. For example, a first vaccine can be administered prior to (e.g., 5 minutes, 15 minutes, 30 minutes, 45 minutes, 1 hour, 2 hours, 4 hours, 6 hours, or 12 hours before), concomitantly with, or subsequent to (e.g., 5 minutes, 15 minutes, 30 minutes, 45 minutes, 1 hour, 2 hours, 4 hours, 6 hours, or 12 hours after) the administration of a second vaccine to a subject. In certain embodiments, the are vaccines can be separated by many days or months, depending on the administration regime for that vaccine. Appropriate administration regimes for combinations of vaccines can be readily devised by the skilled person as required.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed is to be read as being explicitly combined with each of the embodiments, or any permutation of 2 or more of the embodiments disclosed therein.

### Combination Therapy

The method of the present invention also contemplates the use of supplementary active compounds and molecules in combination with ABvac40 vaccine. The supplementary active compounds and molecules can be co-formulated with the ABvac40 vaccine as a unit dosage form, i.e. as a physically discrete unit intended as a unitary dosage for the subject to be treated. Alternatively, the supplementary active compounds and molecules can be presented as a kit-of-parts, and:
administered separately to ABvac40 vaccine, in a phased or sequential dosing pattern; or
co-administered simultaneously from different dosage forms.

For example, the method of the present invention contemplates administering other serum, or plasma-based proteins in combination with ABvac40 vaccine. Serum or plasma proteins within the scope of the present invention include those purified from a suitable plasma source, such as human plasma, and those prepared using recombinant manufacturing techniques. For example, the serum or plasma protein may be selected from the group consisting of Albumin (e.g. ALBUTEIN), Alpha-1 Antitrypsin/Alpha-1 Proteinase Inhibitor (e.g. PROLASTIN), Antithrombin (e.g. THROMBATE III), polyclonal immunoglobulins (IgG, IgA, and combinations thereof), polyspecific immunoglobulins (IgM), C1 esterase inhibitor (e.g. BERINERT), Transthyretin, and combinations thereof.

Exemplary polyclonal immunoglobulins within the scope of the present invention include commercially available polyclonal IgG formulations such as FLEBOGAMMA DIF 5% & 10%, GAMUNEX-C 10%, BIVIGAM 10%, GAMMAGARD Liquid 10%, among others.

Exemplary polyspecific immunoglobulins (IgM) within the scope of the present invention include commercially available immunoglobulin formulations containing polyspecific IgM such as PENTAGLOBIN or TRIMODULIN.

The method of the present invention also provides for administering known neurogenic/neurotrophic compounds and molecules in combination with ABvac40 vaccine. For example, the method of the present invention contemplates administering neurogenic/neurotrophic proteins, peptides, and small molecules alongside ABvac40 vaccine.

Suitable neurogenic and/or neurotrophic compounds and molecules may be selected from the group consisting of BDNF (brain-derived neurotrophic factor; NGF superfamily), GNDF (glial cell line-derived neurotrophic factor), CNTF (cilliary neurotrophic factor-1; neurokine superfamily), PACAP (amino acids 1-38 of pituitary adenylate cyclase-activating polypeptide), Y-27632 and pharmaceutically acceptable salts thereof [trans-4-[(1R)-1-Aminoethyl]-N-4-pyridinylcyclohexanecarboxamide], Fasudil and pharmaceutically acceptable salts thereof [hexahydro-1-(5-isoquinolinyl-sulfonyl)-1H-1,4-diazepine], and combinations thereof.

The skilled person will appreciate that the present invention also contemplates within its scope covalent conjugates of each of the above listed compounds and molecules to the ABvac40 vaccine. Furthermore, it will be appreciated that the present invention also contemplates within its scope recombinant fusion proteins of each of the above listed proteins and peptides with the ABvac40 vaccine.

In one embodiment, the ABvac40 vaccine may constitute at least 20% by weight of the total protein content utilised in the therapeutic method of the present invention. For example, the ABvac40 vaccine may constitute greater than or equal to about 30%, 40%, 50%, 60%, 70%, 75%, 80%, 85%, 90%, 93%, 95%, 96%, 97%, 98%, or 99% by weight of the total protein content utilised in the combination therapy of the present invention.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed is to be read as being explicitly combined with each of the embodiments, or any permutation of 2 or more of the embodiments disclosed therein.

### BRIEF DESCRIPTION OF THE DRAWINGS

Additional features and advantages of the present invention will be made clearer in the appended drawings, in which:

FIG. 1 is the complete amino acid sequence of the vaccine ABvac40 of the present invention.

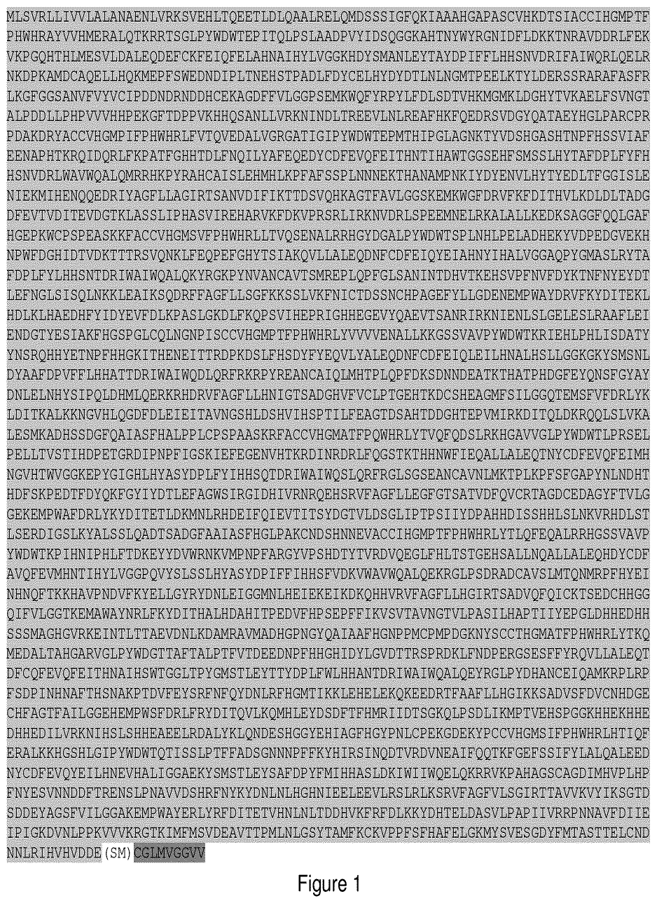

FIG. 2 is a schematic representation of sample sources and testing procedures in the study of the characterization of the immune response elicited by ABvac40.

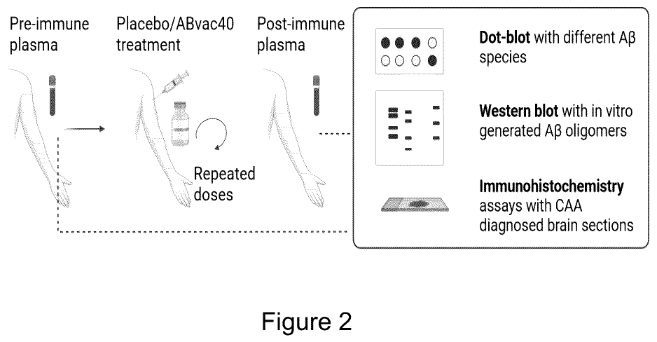

FIG. 3 shows the concentration (ng/ml) of anti-Aβ40 antibodies in CSF, measured by ELISA.

FIG. 4 shows a correlation between the concentration of anti-Aβ40 antibody levels in plasma (μg/ml) and CSF (ng/ml).

FIG. 5 shows the dot blot analysis of plasma samples with different amyloid peptides sequences. A: membrane template; B: pre-immune plasma of vaccinated patient; C: post-immune plasma of vaccinated patient; and D: post-immune plasma of placebo patient;

FIG. 6 shows the western blot analysis of pre-immune (A) and post-immune (B) plasma from a patient of the ABvac40-treated arm. Molecular weights are expressed in kDa;

FIG. 7 shows plasma and CSF immunostaining including all categories of vessels: leptomeningeal and penetrating arteries (arrows in A), arterioles (arrows in B and C) and capillaries (arrowheads in C). Also, some senile plaques are recognized (asterisks in B). Scale bar=50 μm;

FIG. 8 shows the same regions for post-immune (A), pre-immune (B) and pre-adsorbed post-immune (C) plasma staining. Scale bar=100 μm.

FIG. 9 shows the change in Mini Mental State Examination (MMSE) score from baseline to each applicable post-baseline efficacy visit (Part A) analyzed using a Mixed-Model Repeated Measures (MMRM), and the Per-Protocol (PP) cognition (PPc) analysis set.

FIG. 10 shows the change in Trail Making Test A (TMT-A) score from baseline to each applicable post-baseline efficacy visit (Part A) analyzed using a Mixed-Model Repeated Measures (MMRM), and the PPc analysis set.

FIG. 11 shows the percent change in whole brain volume from baseline to each applicable post-baseline efficacy visit (Part A) analyzed using a Mixed-Model Repeated Measures (MMRM), and the PP analysis set.

### DETAILED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### Example 1: Preparation of the KLH-SM-AB(33-40) Conjugates

For preparing these conjugates, KLH was used as a carrier protein, SM as a crosslinking agent and Aβ(33-40) (SEQ ID NO: 1) as an immunogenic peptide (peptide with residues 33-40 of the amyloid peptide to which a cysteine has been added at the N-terminus). Binding took place between the available lysine residues of the KLH and the cysteine added at the N-terminal end of the peptide. In this case, the binding of the crosslinking agent to KLH was done first (KLH activation step), and, in a second step, the immunogenic peptide was added to the activated KLH so that conjugation could take place.

The protocol followed for the foregoing is as follows:
A 250 mM stock solution of SM was prepared by dissolving 100 mg of SM in 680 μl of dry DMSO. Aliquots of said stock solution were made and stored at −20° C.
KLH was dissolved at a concentration of 5 mg/ml in PBS/5 mM EDTA pH 7.4.
16 μL of stock solution of SM were added for each milliliter of KLH that was conjugated.
Said solution was left to react for 2 hours and 30 minutes at room temperature under gentle stirring.
Then, the reaction buffer was changed to remove reaction byproducts and excess unreacted SM. For this purpose, a PD10 column (GE Healthcare; reference 17-0851-01) was used as follows:
   Step a): The column was equilibrated with 25 mL (5 mL, 5 times) PBS (80 mM sodium hydrogenophosphate dihydrate, 20 mM sodium dihydrogenophosphate monohydrate, 100 mM sodium chloride)/5 mM EDTA pH 7.4.
   Step b): 2.5 mL of the already reacted solution were added in the column and said solution was left to penetrate, discarding the eluate.
   Step c): 3.5 mL of PBS were added in the column and the eluate was collected.
   Step d): The column was re-equilibrated with 25 ml (5 mL, 5 times) of 5 mM EDTA pH 7.4, as explained above in step a).
   Steps b) to d) were repeated all the times needed depending on the volume of solution that reacted. After that, the column was rendered equilibrated.
   Step e): The column was stored at 4° C. in order to use it on other occasions with the same peptide conjugate in the same manner. See preceding protocol.
Then the peptide that was to be mixed with activated KLH was calculated. To that end, was taken into account both the molecular weight of the peptide and the molecular weight of KLH, in addition to the active sites therein (measured according to the methods known in the prior art based on measuring absorbance at 343 nm before and after treating the activated KLH solution with dithiothreitol and performing the necessary conversion). For example, for 10 mg of KLH with 1724 active sites:

$$10 / 6725000 ( mean molecular weight of KLH ) = 1.48 \times 10 - 6 mmol of KLH$$

1.48×10−6 mmol of KLH×1724 active sites=2.55×10−3 mmol of peptide required for covering all the active sites. A 3-fold peptide excess was placed to favor the conjugation reaction:
2.55×10-3×3=7.65×10-3 mmol of peptide required.

7.65×10-3× weight molecular of the peptide (834.4 Da)=mg of peptide required (6.38 mg of peptide) (applying the required conversion factors).
Once the peptide/activated KLH ratio (i.e., KLH with bound SM) was determined, the following reaction was prepared: mixture of the peptide with the activated KLH with SM in a suitable proportion. To that end, the peptide was prepared at 6 mg/ml in DMSO and was slowly added to activated KLH. The proportion of DMSO in the final reaction must not exceed 30%. If it were higher than that in any case, PBS/5 mM EDTA pH 7.4 is added until the amount thereof was reduced to values of less than 30% . . . .
The solution was left to react at room temperature and under stirring between 18 and 24 hours.
Finally, the obtained solutions were stored at 4° C. The peptide that has not conjugated with KLH and is therefore free may or may not be removed. If said free peptide is not removed, the peptide concentration in the final product is determined by the total peptide used in the reaction, not only that which is bound to native KLH, and the final reaction volume.

### Example 2: Phase II Clinical Trial Design (Part A)

After undergoing a successful clinical trial Phase I, a second phase of the clinical trial was designed to test the efficacy, safety, tolerability and immune response of ABvac40 on patients with amnestic Mild Cognitive Impairment (a-MCI) or very mild Alzheimer's disease (vm-AD). This was divided in two parts in order to evaluate the effects of the vaccine long and short term, Part A and Part B.

A total of 124 patients with either vm-AD or a-MCI were chosen and divided into two groups at random. One group comprising 62 people received a subcutaneous 1 mL injection of ABvac40 (0.2 mg of Aβ33-40) at the starting point, and then after 4, 8, 12, 16 and a booster at 42 weeks, while the other group of 62 people received a placebo. Two subjects randomized to placebo inadvertently received one dose of ABvac40; therefore, these two subjects were summarized for the safety analysis set in ABvac40 arm. Volunteer patients were chosen randomly from 23 countries in Europe regardless of sex, and neither the patients nor the doctors knew which group got the vaccine and which got the placebo.

There were several objectives to achieve with this second phase of the clinical trial. Regarding safety objectives, a primary safety endpoint was to evaluate the safety and tolerability of repeated doses of ABvac40 in patients with a-MCI or vm-AD. Safety studies were carried out on patients on months 0.5, 1.5, 2.5, 3.5, 4.5, 9, 10.5, 12, 18 and 24, to check for possible emergent adverse effects. Among those, only 26.6% and 26.7% of the patients showed any serious adverse effect on patients that received ABvac40 and those that received the placebo, respectively. Of those percentages, only 4.7% and 13.3% were treatment-related adverse effects.

TABLE 1
Summary of Treatment Emergent Adverse Events (TEAEs) in the
Part A of the ABvac40 Phase 2 study (safety analysis set).

| Treatment Emergent Adverse | ABvac40 | Placebo |
|---|---|---|
| Events (TEAEs) | (N = 64) | (N = 60) |
| Any TEAEs | 58 (90.6%) | 56 (93.3%) |
| Any Treatment-related TEAEs | 29 (45.3%) | 26 (43.3%) |
| Any TEAEs leading to treatment | 4 (6.3%) | 7 (11.7%) |
| discontinuation | | |
| Any TEAEs leading to death | 1 (1.6%) | 1 (1.7%) |
| Any Treatment-emergent Serious | 17 (26.6%) | 16 (26.7%) |
| AE (TESAEs) | | |
| Any Treatment-related TESAEs | 3 (4.7%) | 8 (13.3%) |
| Any TESAEs leading to treatment | 2 (3.1%) | 4 (6.7%) |
| Any TESAEs leading to death | 1 (1.6%) | 1 (1.7%) |
| Any TESAEs of Special Interest | 8 (12.5%) | 9 (15.0%) |
| (TESAESIs) | | |
| ARIA-E | 0 (0.0%) | 0 (0.0%) |
| ARIA-H | 8 (12.5%) | 9 (15.0%) |
| Aseptic meningo-encephalo-myelitis | 0 (0.0%) | 0 (0.0%) |

The overall incidence of adverse events (AEs) was similar between groups. The serious AE were also well distributed between both groups and also the treatment related serious adverse events, and those leading to treatment discontinuation. Regarding the adverse events of special interest, which include ARIA-E, ARIA-H and aseptic meningo-encephalo-myelitis: ARIA-H incidence was equally distributed between groups with no cases of ARIA-E or aseptic meningo-encephalo-myelitis. In conclusion, ABvac40 was safe and well-tolerated.

### Example 3: Phase II Clinical Trial Design (Part B)

Following Part A of the phase II of the clinical trial described above, a second half or Part B was designed. In said Part B, patients that in received placebo in Part A of the trial received a full dose of a subcutaneous 1 mL injection of ABvac40 (0.2 mg of Aβ33-40) at the starting point, and then after 4, 8, 12, 16 and a booster at 42 weeks, just like the ABvac40 group on Part A. Meanwhile, patients that received the vaccine in Part A received placebo on weeks 0, 4, 8, 12 and 42, but received a booster on week 16.

This second part of the clinical trial phase II showed similar results to those of Part A in terms of tolerability and safety, as only a single mild ARIA-H was recorded on patients receiving ABvac40, and thus confirmed that ABvac40 is a safe vaccination, well tolerated in most cases.

### Example 4: Effects of ABvac40 in Increasing Immunogenicity Against Aβ40

In order to evaluate whether ABvac40 was working as intended, patients from the clinical trial described above were taken samples to measure several parameters.

First, the levels of anti-Aβ40 antibody signal were tested. To do that, optical density (OD) of the samples from ABvac40-receiving patients was measured by ELISA and compared to those from the placebo group.

The results showed that the levels of anti-Aβ40 in the ABvac40 group were significantly higher than those in the placebo group, showing that the main objective of increasing anti-Aβ40 antibody levels was successful.

When compared the levels of anti-Aβ40 antibody in patients after the first complete immunization (Part A) to the levels shown when received the booster on week 16 of Part B, it was shown that said booster elicited a 4-fold increase on maximum antibody levels. This increase demonstrates that there are memory B and T cells that matured during the original immunization and can be stimulated upon detection of the Aβ40 antigen.

Lastly, it was observed that plasma levels of total Aβ40 increased along with the ABvac40 doses, mirroring the levels of anti-Aβ40 antibody. However, although the levels of total Aβ40 increased, the levels of free Aβ40 diminished over time upon receiving a dose of the vaccine, and even more when received the booster from Part B of the clinical trial.

Therefore, it is clear that ABvac40 effectively works as an immunization agent in plasma and achieves a prolonged immune memory, therefore reducing the levels of free Aβ40 and increases the response when stimulated again in a later time point, proving that it can be effective as a long-term protection against newly-formed Aβ40 deposits.

These same effects were, although not as pronounced, also observed in cerebrospinal fluid, demonstrating a correlation (p value<0.0001) between the increase of anti-Aβ40 antibody levels in plasma and the increase of anti-Aβ40 antibody levels in CSF.

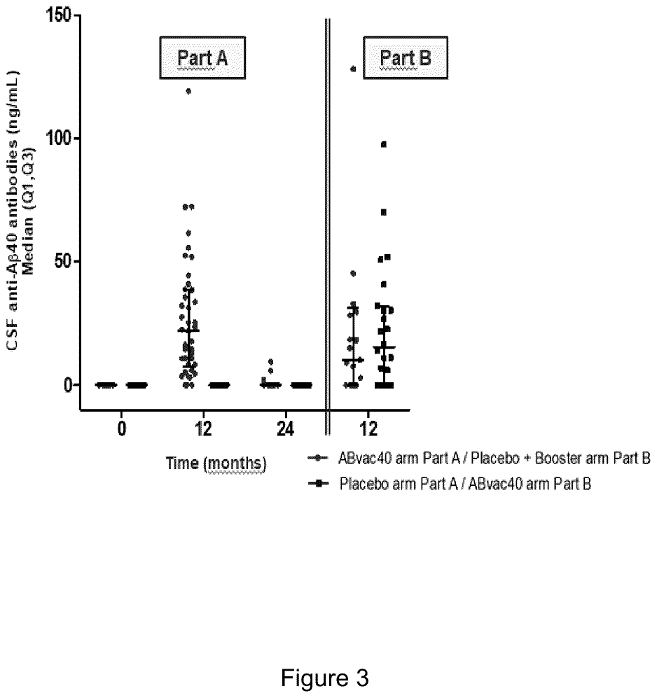

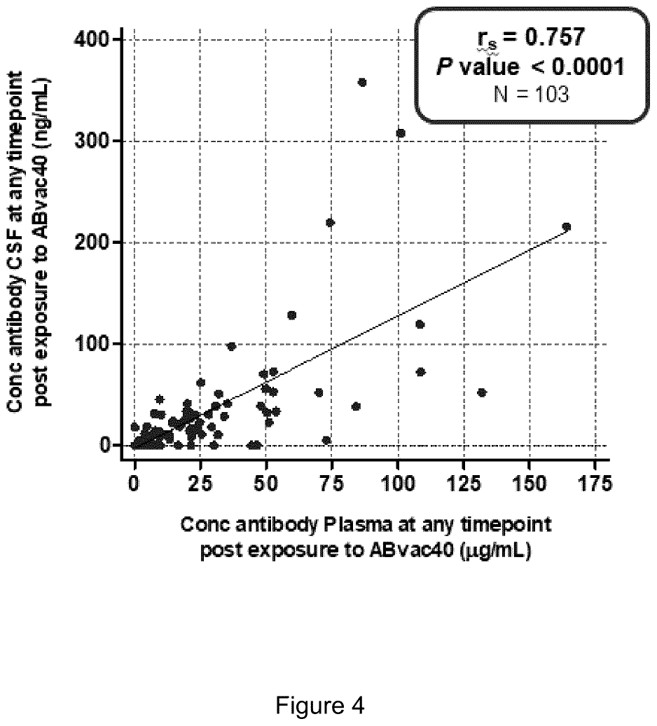

As shown in FIG. 3 anti-Aβ40 antibodies were detected in CSF, with a CSF: plasma ratio of 0.1% similar to the ratio reported by other immunotherapies. 8 months after the patients received a booster in Part B, antibodies were still detected in CSF. In FIG. 4 a correlation between the concentration of anti-Aβ40 antibody levels in plasma (μg/ml) and CSF (ng/ml) is shown. The levels of antibodies in plasma and CSF were significantly correlated (Spearman rho is used for the correlation analysis).

### Example 5: Reactivity Against Vascular Amyloid Deposition in Human Brains with Cerebral Amyloid Angiopathy (CAA)

Firstly, the reactivity of the antibodies raised by ABvac40 to target different forms of Aβ was analysed by immunoblotting. Dot blot assays were conducted using synthetic AB peptides of different lengths, truncated at both the N-terminal and C-terminal ends. Nitrocellulose membranes were probed with pre-immune and post-immune (after five ABvac40 or placebo inoculations) plasma samples from three patients from placebo arm and patients from ABvac40 arm.

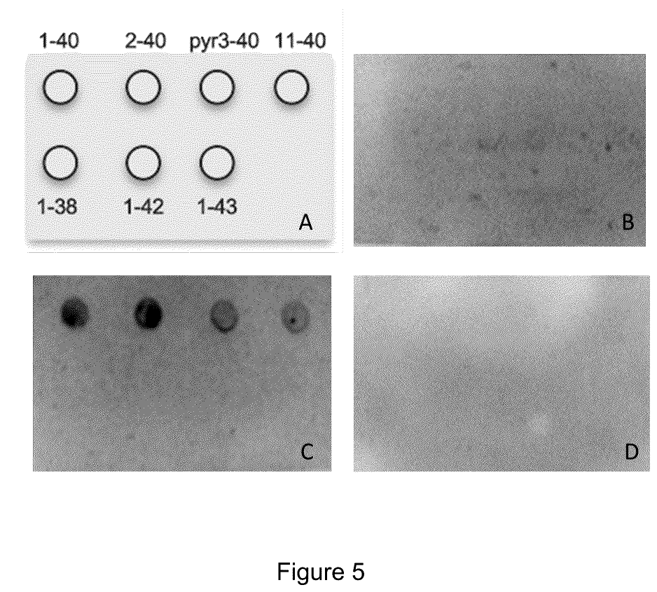

Plasma samples from patients treated with ABvac40 exhibited specific dot-blot immunoreactivity against Aβx-40 peptides, whereas they did not bind to amyloid peptides ending at amino acids 38, 42 or 43 (FIG. 5). Neither the plasma collected before treatment nor the plasma from patients who received placebo demonstrated recognition of any of the peptides examined in the study.

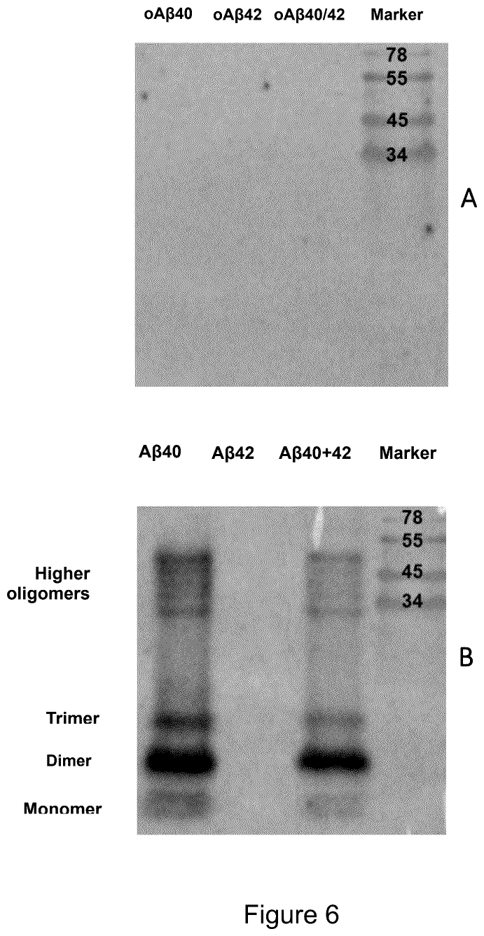

On the other hand, Western blot assays were performed using in vitro-generated oligomers, composed of either Aβ40, Aβ42, or a combination of Aβ40/Aβ42 (in a ratio 9/1). The membranes were incubated with plasma samples from three ABvac40-treated patients (pre-immune and after seven doses of ABvac40). As shown in FIG. 6, Western blot analysis of oligomers of Aβ40 (0Aβ40), Aβ42 (0Aβ42) and a combination of Aβ40 and Aβ42 (oAβ40/42) revealed that antibodies in plasma samples from ABvac40-treated patients recognized Aβ40 in various forms, including monomers, dimers, trimers and higher oligomers. However, these antibodies did not exhibit any reactivity towards Aβ42, either in its monomeric form or aggregated states.

In addition, immunohistochemistry analyses were carried out on post-mortem human brain paraffin-embedded sections from three patients diagnosed with CAA and three healthy controls (provided by CIEN Foundation Tissue Bank, Madrid). These histological sections were incubated with three post-immune plasma samples (after seven doses of ABvac40), using pre-immune plasma of the same patients as negative control and pre-adsorbed post-immune plasma as a control of specificity.

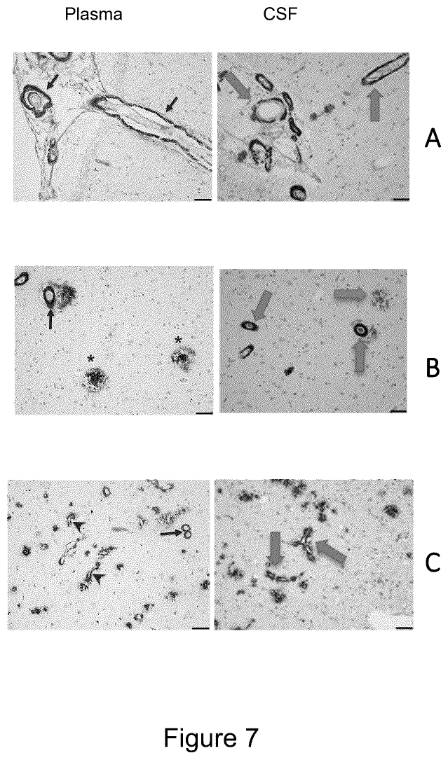

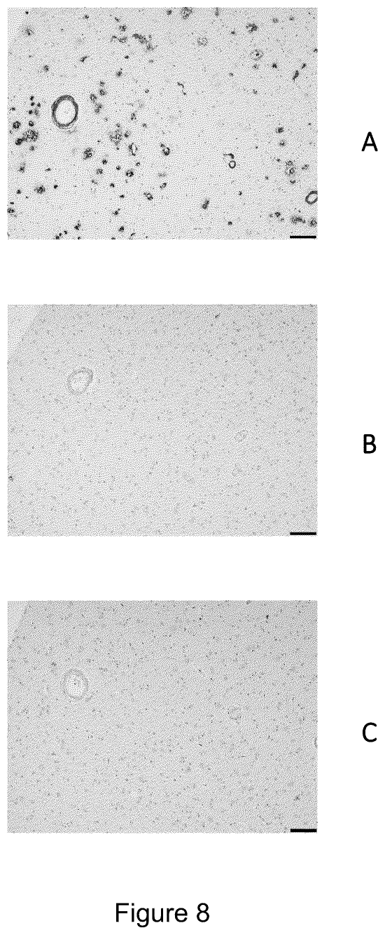

Staining of occipital brain tissue sections from the three neuropathologically diagnosed CAA patients with all ABvac40-treated plasma samples tested revealed strong immunoreactivity against Aβ vascular deposits in all types of vessels (leptomeningeal and penetrating arteries, arterioles and capillaries). Additionally, some senile plaques were also recognized (FIG. 7). These signals were absent when using plasma samples obtained prior immunization. The staining specificity was confirmed through pre-adsorption of post-immune plasma with the specific antigen (FIG. 8).

This study demonstrates the specificity of plasma antibodies triggered by ABvac40, which recognize the C-terminal region of Aβx-40 peptides in different aggregated states (monomeric, oligomeric, and brain deposits). Binding of ABvac40-elicited antibodies to vascular and parenchymal amyloid brain deposits could have an important role in the mechanism of action of ABvac40. We hypothesize that the interaction of anti Aβ40 antibodies with vascular amyloid could lead to a reduction of amyloid deposition in cerebral vessels. This could enhance the clearance of brain waste through perivascular spaces, potentially contributing to cognitive improvement, while maintaining an excellent safety profile as demonstrated previously.

### Example 6: Secondary Exploratory Efficacy Endpoints (Part A): MMSE

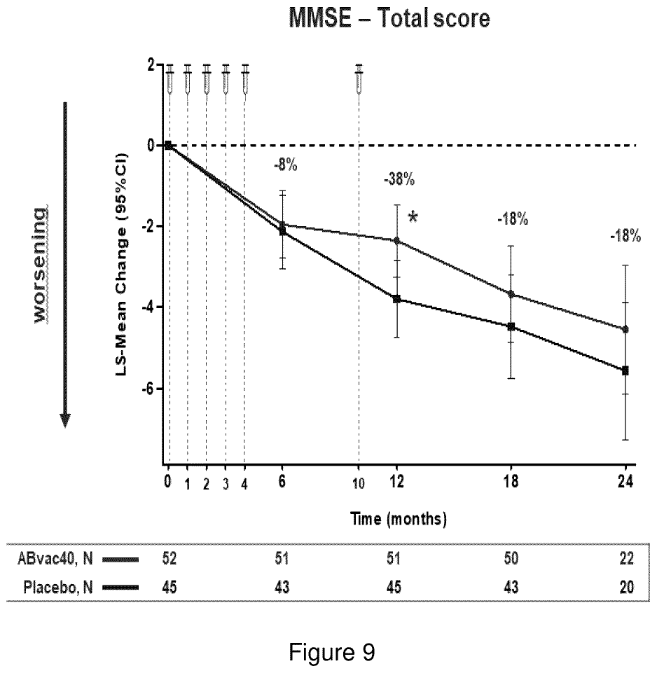

As shown in FIG. 9, the change in Mini Mental State Examination (MMSE) score from baseline to each applicable post-baseline efficacy visit (Part A) was analyzed using a Mixed-Model Repeated Measures (MMRM), and the PPc analysis set. MMSE is an 11-question measure that tests five areas of cognitive function: orientation, registration, attention and calculation, recall, and language. MMSE score ranges from 0 to 30, with lower scores indicating worst cognition.

ABvac40 group showed a maximum slowing of disease progression of 38% compared to placebo as evidenced by changes in MMSE score starting at month 6, resulting in a statistically significant difference at month 12 (p value<0.05).

### Example 7: Secondary Exploratory Efficacy Endpoints (Part A): TMT-A

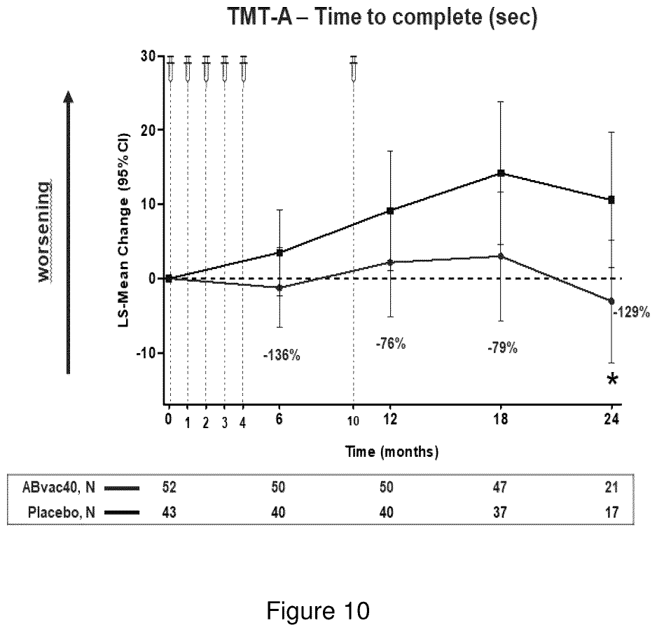

As shown in FIG. 10, the change in Trail Making Test A (TMT-A) score from baseline to each applicable post-baseline efficacy visit (Part A) was analyzed using a Mixed-Model Repeated Measures (MMRM), and the PPc analysis set: TMT-A is a measure in which the patient is instructed to connect a set of 25 numbers (from 1 to 25) in sequential order as quickly as possible while still maintaining accuracy. The lower timings mean a better outcome.

Shorter time to complete TMT-A was observed in ABvac40 group vs placebo, reaching significance at month 24 (p value<0.05).

### Example 8: Secondary Exploratory Efficacy Endpoints (Part A): Whole Brain Volume

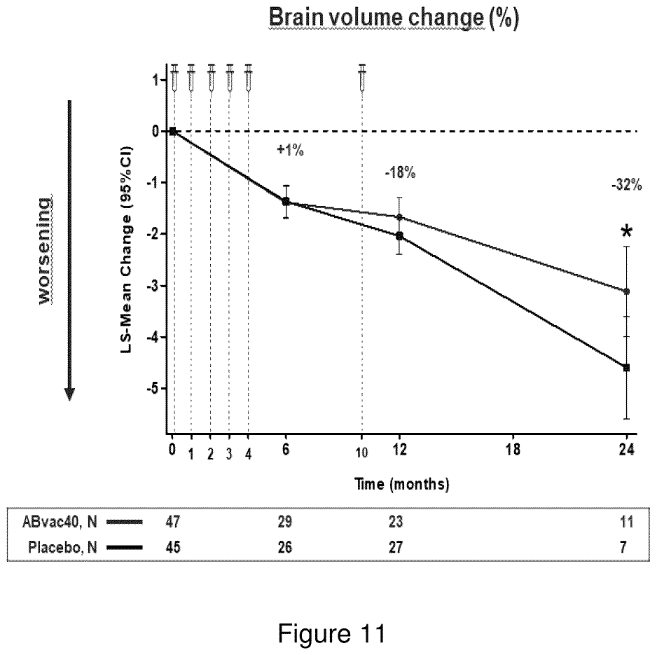

As shown in FIG. 11, the percent change in whole brain volume from baseline to each applicable post-baseline efficacy visit (Part A) was analyzed using a Mixed-Model Repeated Measures (MMRM), and the PP analysis set: Whole brain volume was measured by MRI. The more positive the change in total brain volume means a better outcome.

At months 12 and 24, volumetric MRI showed lesser progression in whole brain atrophy in 5 the ABvac40 group versus placebo, reaching statistical significance at month 24 (p value<0.05).

1. A vaccine composition for use in the prevention and/or treatment of cerebral amyloid angiopathy (CAA), said vaccine composition comprising an amyloid beta 33-40 (Aβ33-40) peptide with an additional cysteine on the N-terminal end conjugated with the carrier protein keyhole limpet hemocyanin (KLH), using as the crosslinking agent the maleimidobutiric acid N-hydroxysuccinimide ester (SM) and an aluminum hydroxide gel.

2. Composition for use, according to claim 1, wherein the aluminum hydroxide gel is Al(OH)₃.

3. Composition for use, according to any of claim 1 or 2, wherein said the vaccine composition comprises the CysAβ33-40 peptide at a concentration between 50 μg/ml and 500 μg/ml.

4. Composition for use, according to claim 3, wherein the CysAβ33-40 peptide is at a concentration of 200 μg/ml.

5. Composition for use, according to any of the preceding claims, wherein said vaccine composition is administered by an administration route selected from the group consisting of intravenous, subcutaneous, intramuscular, intradermal, intraperitoneal, intrapulmonary, intranasal, oral, rectal, and combinations thereof.

6. Composition for use, according to claim 5, wherein said vaccine composition is administered subcutaneously.

7. Composition for use, according to any of the preceding claims, wherein said vaccine composition is administered at multiple intervals as part of a multiple-dosage regimen.

8. Composition for use, according to claim 7, wherein said multiple dosing regimen comprises multiple portion doses administered on weeks 0, 4, 8, 12 and 16.

9. Composition for use, according to claim 8, wherein said multiple dosing regimen further comprises a booster administered on week 42.

10. Composition for use, according to claim 9, wherein said multiple dosing regimen further comprises a booster administered on week 58.

11. Composition for use, according to any of the preceding claims, wherein the vaccine composition is administered at individual doses of about 0.5 ml to about 2 ml per interval as part of a multiple-dosage regimen.

12. Composition for use, according to any of the preceding claims, wherein supplementary active compounds and molecules are used in combination with said vaccine composition.

13. Composition for use, according to claim 12, wherein said supplementary active compounds or molecules are plasma-based proteins.

14. Composition for use, according to claim 13, wherein said plasma-based proteins include those purified from a suitable plasma source, and are selected from the group consisting of albumin, alpha-1 antitrypsin/alpha-1 proteinase inhibitor, antithrombin, polyclonal immunoglobulins such as IgG, IgA, and combinations thereof, polyspecific immunoglobulins such as IgM, C1 esterase inhibitor, transthyretin, and combinations thereof.

15. Composition for use, according to claims 12 to 14, wherein said supplementary active compounds or molecules are neurogenic/neurotrophic compounds and molecules.

16. Composition for use, according to claim 15, wherein said neurogenic and/or neurotrophic compounds and molecules are selected from the group consisting of brain-derived neurotrophic factor, glial cell line-derived neurotrophic factor, cilliary neurotrophic factor-1, amino acids 1-38 of pituitary adenylate cyclase-activating polypeptide, Y-27632 pharmaceutically and acceptable salts thereof [trans-4-[(1R)-1-Aminoethyl]-N-4-pyridinylcyclohexanecarboxamide], fasudil and pharmaceutically acceptable salts thereof [hexahydro-1-(5-isoquinolinyl-sulfonyl)-1H-1,4-diazepine], and combinations thereof.

17. Composition for use, according to any of the preceding claims, wherein said vaccine composition constitutes at least 20% by weight of the total protein content utilised in the therapeutic method.

18. Composition for use according to any of the preceding claims wherein said vaccine composition is used in combination with one or more vaccines for the prevention and/or treatment of CAA or other associated neurological disorders, in particular Alzheimer's Disease.
